(19) United States
(12) Patent Application Publication
(10) Pub. No.: US 2020/0215190 A1
(43) Pub. Date: Jul. 9, 2020

(54) Zinc phthalocyanine (ZnPC) and Perylene (PY) Co-Loaded Multifunctional Nanoparticles for Photodynamic Therapy (PDT)

(71) Applicant: The Government of the United States of America, as represented by the Secretary of the Navy, Arlington, VA (US)

(72) Inventors: Okhil Nag, Alexandria, VA (US); Jawad Naciri, Arlington, VA (US); Jeff Erickson, Bethesda, MD (US); James B. Delehanty, Washington, DC (US)

(21) Appl. No.: 16/793,191

(22) Filed: Feb. 18, 2020

Related U.S. Application Data
(63) Continuation of application No. PCT/US18/00220, filed on Aug. 16, 2018.
(60) Provisional application No. 62/546,778, filed on Aug. 17, 2017.

(51) Int. Cl. A61K 41/00; A61K 31/315; A61K 31/015; A61K 9/51; A61K 47/10

(52) U.S. Cl. CPC A61K 41/0071; A61K 31/315; B82Y 40/00; A61K 9/513; A61K 47/10; A61K 31/015

(57) ABSTRACT

A liquid crystal nanoparticle (LCNP)-based system allows for the encapsulation and targeted delivery of Zinc (II) phthalocyanine (ZnPC) to the plasma membrane bilayer of living cells for photodynamic therapy (PDT). The formulation comprises LCNPs that are loaded in their hydrophobic core with perylene (PY) and ZnPC. In embodiments, the LCNP surface is functionalized with Poly(ethylene glycol)-cholesterol conjugates (PEG-Chol) and/or another material enabling targeting the particle to the cellular membrane. This can improve cell killing via reactive oxygen species (ROS) generation as it allows for the localized ROS-mediated peroxidation of lipids in the membrane bilayer.

### CROSS-REFERENCE TO RELATED APPLICATIONS

This Application claims the benefit of patent application nos. 62/546,778 filed on Aug. 17, 2018 and PCT/US18/00220 filed on Aug. 16, 2018, the entirety of each of which is incorporated herein by reference.

### BACKGROUND

Photodynamic therapy (PDT) is a clinically approved method for tumor ablation. PDT includes two elements: (1) a PDT drug (also known as a photosensitizer (PS)) and (2) light for irradiating the PS. During treatment, the photoexcited PS produces reactive oxygen species (ROS) to damage and kill cells in the vicinity of the PS. Generation of excessive ROS in tissue leads to cell death and tumor destruction. Most current PS molecules (e.g., porphyrin, chlorin, porphycene, etc.) are macrocyclic compounds which suffer from poor water solubility, with a tendency to aggregate via π-π stacking in polar media such as aqueous solutions. This results in reduced effective concentrations and renders the PS molecules photodynamically inactive or less effective when administered to the tissues. Zinc (II) phthalocyanine (ZnPC), a derivative of porphyrin, is a promising PS due to its high ROS quantum yield, also suffers from the above limitations when administered to the body. Although there are several synthetic approaches (including the addition of charged, water-soluble substituents) to render the ZnPC more stable in aqueous media, none of these have satisfactorily solved the problem without the further addition of surfactants or organic solvents (which can be toxic). Further, the introduction of charged substituents reduces the ability of ZnPC to interface with cellular structures such as the plasma membrane. Additional issues are the lack of subcellular specificity of ZnPC once internalized into cells which reduces its therapeutic efficacy. Recent developments of nanoparticle-based formulation of PS molecules have addressed some of the solubility/delivery issues for ZnPC. However, the self-aggregation even inside the NPs and specific delivery to the subcellular location remains challenging. Another limitation of currently employed PS molecules is their rather modest two photon absorption (TPA) which limits their use in deeper tissue applications

A need exists for improved photosensitizers for photodynamic therapy.

### BRIEF SUMMARY

Described herein is a hybrid liquid crystal nanoparticle (LCNP)-based delivery system for the encapsulation and targeted delivery of Zinc (II) phthalocyanine (ZnPC) to the plasma membrane bilayer of living cells. The formulation comprises LCNPs that are loaded in their hydrophobic core with perylene (PY) and ZnPC. In embodiments, the LCNP surface is functionalized with Poly(ethylene glycol)-cholesterol conjugates (PEG-Chol) and/or another material enabling targeting the particle to the cellular membrane. Targeting the LCNPs to the plasma membrane with the PEG-Chol moiety improves cell killing via reactive oxygen species (ROS) generation as it allows for the localized ROS-mediated peroxidation of lipids in the membrane bilayer.

Co-loading the PY and ZnPC into the LCNP achieves the following objectives: (1) optically exciting the ZnPC via fluorescence resonance energy transfer (FRET) by using the photoexcited PY as the energy donor, (2) preventing the self-aggregation of ZnPC inside the particle core, (3) allowing tracking/imaging of the particles or labelled tissue using fluorescence-based imaging, and (4) improving the two photon absorption (TPA) of the ZnPC by using the PY dye (which has a large TPA) as energy donor.

### BRIEF DESCRIPTION OF THE DRAWINGS

FIGS. 1A through 1E illustrate zinc phthalocyanine (ZnPC), or hybrid loaded (PY-ZnPC) loaded liquid crystal nanoparticles (LCNPs). FIG. 1A shows the chemical structures of the compounds utilized to prepare the LCNPs: (top row) an acrylate liquid crystal cross-linking agent (DACTP11), and (bottom row) a carboxyl-terminated polymerizable surfactant (AC10COONa). FIGS. 1B and 1C show the chemical structures of the (B) photodynamic therapy (PDT) drug, zinc phthalocyanine (ZnPC) and (C) perylene (PY) chromophore. FIGS. 1D and 1E are schematic representation and photographs of various LCNP suspensions used herein: (D) ZnPC-LCNP, and (E) PY-ZnPC-LCNP.

FIG. 2A is a schematic of the ZnPC loaded LCNP and its conjugation to cholesterol-terminated poly(ethylene glycol) (PEG-Chol) via EDC coupling. Addition of PEG-Chol to the ZnPC-LCNP mediates preferential binding of the NP to the plasma membrane. FIG. 2B shows results of gel electrophoresis analysis of LCNPs in 1% agarose gel. The arrow on the left represents the line of wells where samples were loaded. Unconjugated LCNPs (lanes 1 and 3) migrate further towards the cathode (+) compared to the conjugated LCNPs (lanes 2 and 4). The inset shows high contrast image for samples 1 and 2.

FIGS. 3A-3D show the spectral properties of the PEG-Chol conjugated LCNPs. FIGS. 3A shows normalized absorption spectra of LCNPs in 0.1× PBS (pH 7.4) (solid line) and digested LCNPs (dried and reconstituted in chloroform:methanol (3:1, v:v)) (dashed line). FIGS. 3B shows normalized absorption spectra of ZnPC solution in chloroform:methanol (3:1, v:v). FIGS. 3C shows fluorescence spectra of the LCNPs excited at 533 nm and 638 nm. Samples were prepared by diluting stock solutions of LCNPs to a final ZnPC concentration of 4.0 μM. FIGS. 3D is an inset view of select region of FIG. 3C showing spectral detail in the region of fluorescence intensity of 0-5000 a.u.

FIGS. 4A and 4B provide results of the quantification of generation of reactive oxygen species (ROS) by LCNPs. A fluorescent probe (CellROX ROS,ThermoFisher Scientific) was used to quantify the photoexcited ROS generated by various LCNP formulations. In FIG. 4A, a bar graph shows the increase in fluorescence intensity of the ROS probe after irradiation with the lasers (532 nm or 638 nm) for the time indicated. Each LCNP sample was prepared by mixing constant concentrations of ZnPC (4.0 μM) and ROX probe (10.0 μM) in 0.1× PBS (pH 7.4). The sample of ZnPC free in solution (ZnPC-free) was prepared in DMSO. FIG. 4B shows time-resolved ROS generation of PY-ZnPC-LCNP upon direct excitation (638 nm), or in FRET excitation configuration (532 nm) (FRET from PY to ZnPC). The data for each sample was derived from fluorescence spectra of three independent experiments (n=3±SEM) and normalized to the emission intensity before laser irradiation.

FIG. 5 provides confocal laser scanning microscopy (CLSM) images showing labeling of the plasma membrane with LCNPs in HEK293 T/17 cells. Shown are differential interference contrast (DIC) and confocal fluorescence images of live cells stained with PEG-Chol-conjugated PY-LCNP, ZnPC-LCNP and PY-ZnPC-LCNP. The FITC (green; excitation 488 nm, emission 500-550 nm) and TRITC (red; excitation 543 nm, emission 570-620 nm) emission correspond to PY or/and ZnPC in the plasma membrane-associated LCNPs. ZnPC-LCNP were excited with 488 nm and imaged through TRITC channel. The samples were prepared by incubating the cells with ˜50 nM of each LCNP formulation, corresponding to concentrations of PY and ZnPC of 35.0 μM and 6.0 μM, respectively. Scale bar, 20 m.

FIGS. 6A-6C show cellular proliferation results after LCNP-mediated photodynamic therapy (PTD) treatment. HeLa cells were labeled with Chol-PEG-conjugated LNCP formulations and irradiated as indicated. FIG. 6A displays an analysis of cell migration by scratch wound assay. Shown are the representative DIC images of HeLa cells taken at 0, 24 and 48 h after PTD treatment. The samples were treated with LCNPs with or without ZnPC (6.0 μM when present) and irradiated with 638 nm or 532 nm laser for 30 min before scratching with 200 μL pipette tip. The calculated total energy dose that the cells exposed during the treatment is approximately 16.0 J/cm² for 532 nm and ˜13 J/cm² for 638 nm. Minimal cell migration into the scratched area is observed with the sample PY-ZnPC-LCNP excited at 532 nm. Images were acquired with a 10× objective. FIG. 6 B illustrates the quantification of the scratched area that remained open at the indicated time points after PDT treatment. The open areas were calculated by drawing region of interest (ROI) on the images (n=5-7 from 2 independent experiments) along the edge of the migrated/proliferated cells. FIG. 6C presents DIC images of HeLa cells stained with trypan blue 3 h after the PDT treatment with LCNPs coupled with laser excitation. The cell sample treated with PY-ZnPC-LCNP excited at 532 nm shows significant staining with trypan blue indicating compromised plasma membrane. Images were acquired with a 20× objective.

FIG. 7 displays the quantification of cytotoxicity by MTS assay. LCNPs (ZnPC 6.0 μM) were incubated on HeLa cell monolayers for 20 min and then removed. Cells were washed and treated with the lasers for 30 min and cultured in growth medium for 72 h prior to MTS assay. The calculated total energy dose that the cells exposed during the treatment is approximately 16.0 J/cm² for 532 nm and ˜13 J/cm² for 638 nm. Cell viability (%, n=3±SEM) is obtained by normalizing MTS absorbances to the control sample for which cells were not treated with both LCNPs or lasers.

FIG. 8 shows the visualization of HeLa cell morphology upon PTD treatment with LCNPs. Shown are the merged CLSM images channels DAPI (blue) and FITC (green) of fixed Hela cells after PDT treatment. LCNPs treated or untreated (control) cells were irradiated for 30 min as indicated and nuclei and actin were stained with DAPI and F-actin, respectively. The calculated total energy dose that the cells exposed during the treatment is approximately 16.0 J/cm2 for 532 nm and ˜13 J/cm2 for 638 nm. The cells treated with PY-ZnPC-LCNP excited at 532 nm show significant nuclear condensation and structural disorganization of the actin network. Scale bar, 20 μm.

### DETAILED DESCRIPTION

Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Overview

A multifunctional liquid crystal nanoparticle (LCNP) can be loaded with a dye (such as perylene (PY)) and a PDT drug (such as Zinc (II) phthalocyanine, termed ZnPC) as an energy donor and acceptor, respectively, for PDT treatment. This hybrid LCNP includes (1) a hydrophobic core where the hydrophobic molecules PY and ZnPC are incorporated during synthesis and (2) a carboxylate functionalized surface where a ligand (PEGylated-cholesterol, PEG-Chol) is covalently conjugated to mediate attachment of the LCNP to the plasma membrane of cells. Tight packing of the PY and ZnPC in the LCNP core allows the NP to efficiently generate reactive oxygen species (ROS) via FRET from PY to ZnPC, while PEG-Chol facilitates the close association of the LCNP with the plasma membrane. FRET excitation of the PY-ZnPC pair surprisingly generates significantly greater reactive oxygen species ROS (3.5-fold) in cells labeled with the PDT LCNPs compared to when the ZnPC is excited directly, thus making it a novel and more efficient NP-based PDT treatment.

### EXAMPLES

As detailed below, the LCNPs were delivered to the plasma membrane of living cancer cells that are subjected to PDT treatment with direct (638 nm) and FRET (532 nm) excitation of the ZnPC PDT moiety for 30 min. Both cellular proliferation and migration were significantly reduced when cells were treated with PY-ZnPC-LCNP via FRET excitation of ZnPC. FRET excitation of the ZnPC reduces cellular viability 83% compared to a reduction in cellular viability of only 95% when excited in direct excitation mode.

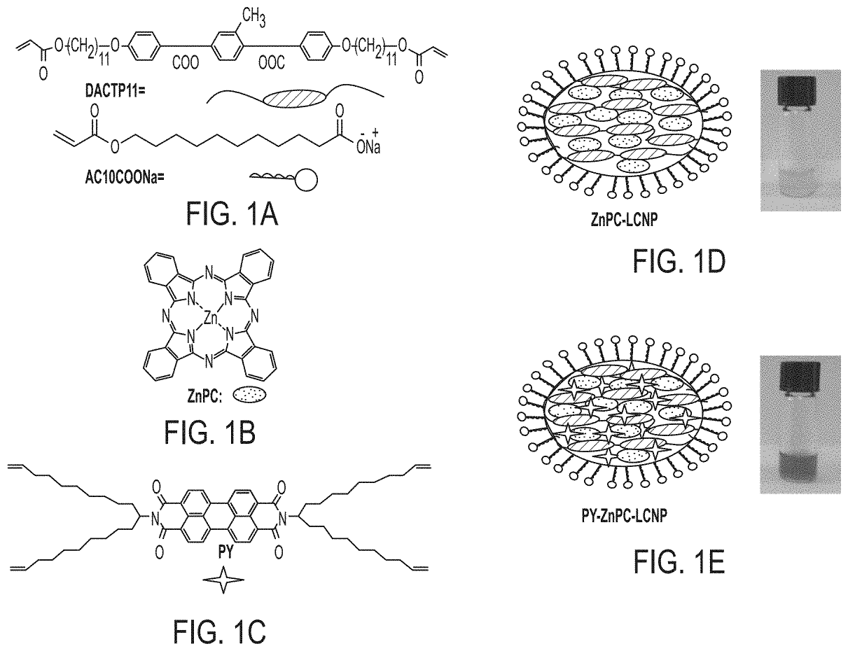

FIG. 1 shows a schematic representation of the components and the LCNP used. In contrast to other commonly used surfactant-based NPs, the polymerizable liquid crystalline agent used herein is covalently crosslinked such that it provides a hydrophobic network with added stability. This helps to reduce the aggregation of the ZnPC moiety. The carboxylate moiety on the LCNP surface provides stability in aqueous media and presents a functional handle for attachment of cell-targeting ligands.

Additional details regarding the preparation and modification of LCNPs can be found in “Hybrid Liquid Crystal Nanocarriers for Enhanced Zinc Phthalocyanine-Mediated Photodynamic Therapy” by Okhil K. Nag et al., Bioconjugate Chemistry Article ASAP online publication DOI: 10.102/acs.bioconjchem.8b00374 as well as the first group of references at the end of this specification, all of which are incorporated herein by reference for the purposes of teaching the preparation and modification of LCNPs.

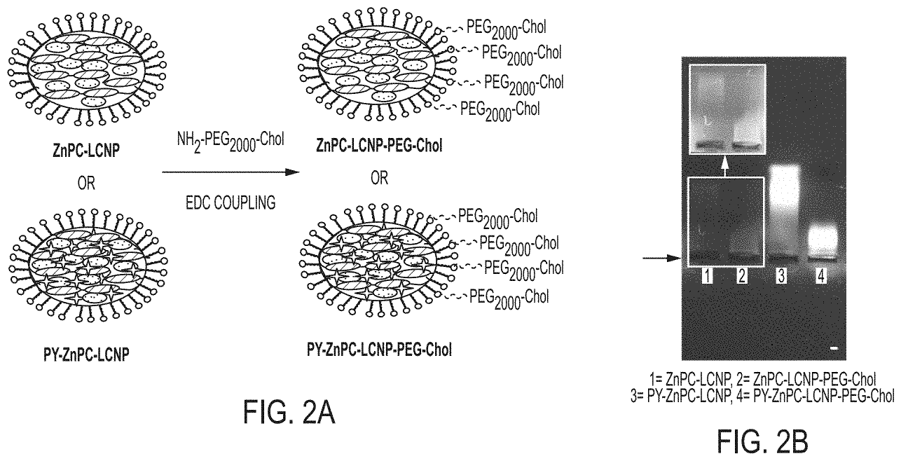

The bare LCNPs were further surface modified with PEG₂₀₀₀-Chol via EDC coupling. FIG. 2 shows the schematic for the EDC reaction and characterization by gel electrophoresis. As anticipated, negatively charged, unconjugated LCNPs showed clear and strong mobility toward the cathode (+). However, after functionalization with PEG-Chol (and the resulting consumption of negatively changed free carboxyl groups) the LCNPs-PEG-Chol particles exhibited minimal migration toward the cathode, confirming the decrease in negative surface charges. This data provides indicates that the process resulted in the successful conjugation of PEG-Chol onto the NP surface.

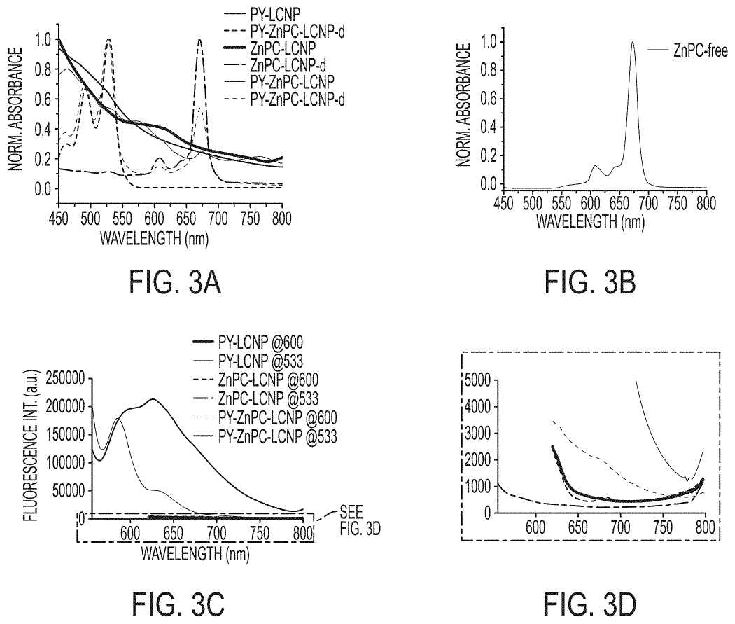

Successful encapsulation of ZnPC in LCNPs and co-encapsulation of PY and ZnPC in the hybrid LCNP were confirmed by UV-vis spectroscopy (FIGS. 3A and B) before and after digestion of the LCNPs with mixed organic solvents (chloroform/methanol (3:1). After digestion of the LCNPs PY and ZnPC show the characteristic λ_{abs} at about 530 nm and 672 nm, respectively. This suggests that PY and ZnPC maintain their chemical integrity and optical properties during the synthesis of the LCNPs. In addition, UV-vis spectra provide a calculated ratio PY:ZnPC of 6:1 in the core of the LCNPs. Steady state fluorescence measurements (FIG. 3B) were performed to determine the efficiency of FRET between the PY donor and ZnPC acceptor. The fluorescence spectra were measured by exciting LCNPs using two different wavelengths: 600 nm (direct excitation of ZnPC) and 533 nm (direct excitation of PY). As observed in the spectra, the FRET excitation of ZnPC provides more than 75 and 150 times higher fluorescence emission intensity compared to the direct excitation of ZnPC in the PY-ZnPC-LCNP and ZnPC-LCNP formats, respectively. These results provide strong evidence that incorporation of PY in the ZnPC-LCNP not only provide a means of indirectly and efficiently exciting ZnPC via FRET, but also significantly prevents aggregation induced self-quenching of ZnPC inside the particle.

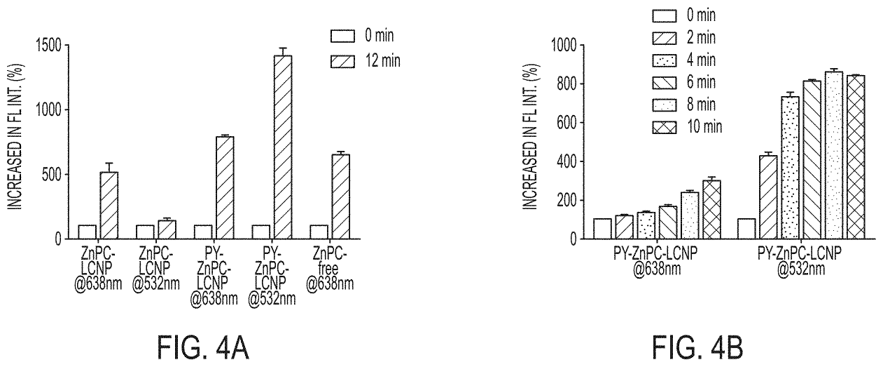

ROS generation efficiency of the LCNPs was studied with a fluorescence probe that shows an increase in fluorescence emission upon generation of ROS. As show in FIG. 4, upon 12 min irradiation the fluorescence emission intensity of the ROS probe increased ˜1400% when PY-ZnPC-LCNP samples were excited at 532 nm via FRET compared to ˜500% and ˜800% to directed excitation at 638 nm of ZnPC in the PY-ZnPC-LCNP and ZnPC-LCNP, respectively. The high ROS generation efficiency of PY-ZnPC-LCNP at 532 nm versus 638 nm is further confirmed by time resolved excitation of the probe, as shown in FIG. 4B.

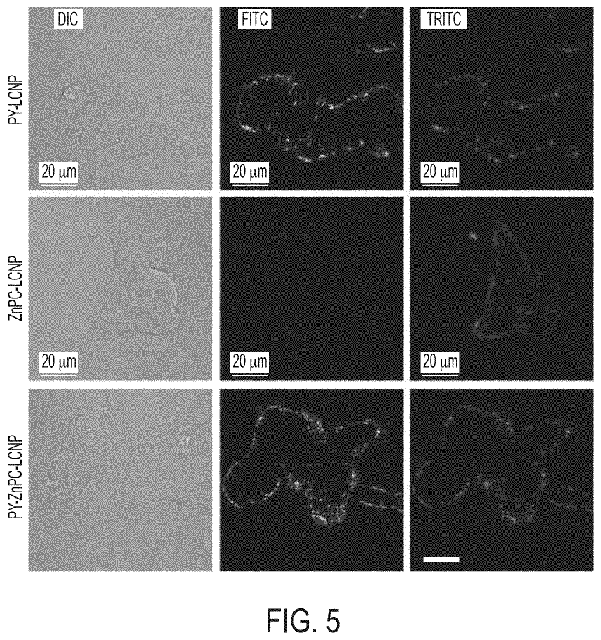

Fluorescence imaging was used to confirm the successful labeling of the plasma membrane of cells with the LCNPs-PEG-Chol. As evidenced by the fluorescence micrographs in FIG. 5, the plasma membrane of HEK 293T/17 cells were labelled with LCNPs where the LCNPs were tracked by the fluorescence signal coming from the PY dye which appears in both the FITC and TRTC channels due to the dye's broad emission spectrum.

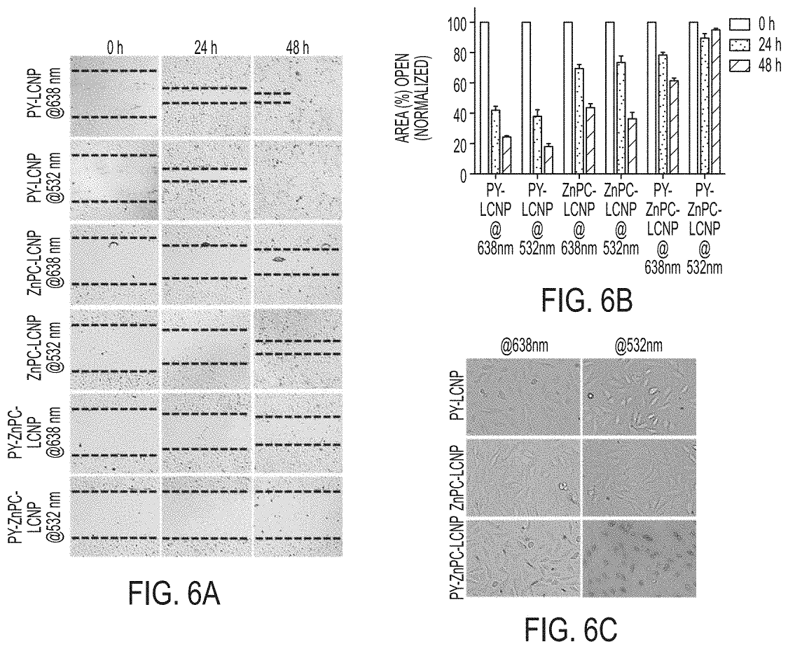

Given efficient ROS generation and the controlled membrane-specific delivery of PY-ZnPC-LCNPs, the ability the LCNPs to modulate cellular migration/proliferation after irradiation with light was examined. For this, a scratch wound assay was performed after labeling the cells with LCNPs, exposing the labeled cells to excitation light, and then culturing in standard incubation condition for 48 h (FIG. 6A). FIG. 6B shows the quantified scratched area (%) that remains unfilled with the cells at indicated time point after the treatment. It is apparent that after 48 h ˜95% of the area remains open (indication of cell killing) for cells labeled with PY-ZnPC-LCNPs and excited at 532 nm (FRET configuration) compared to ˜61% for PY-ZnPC-LCNPs excited at 638 nm, ˜36% for ZnPC-LCNPs excited at 532 nm, and ˜43% for ZnPC-LCNPs excited at 532 nm. Minimal open scratched area (˜20%) remained unfilled for cells treated with control PY-LCNPs excited at either 638 nm or 532 nm.

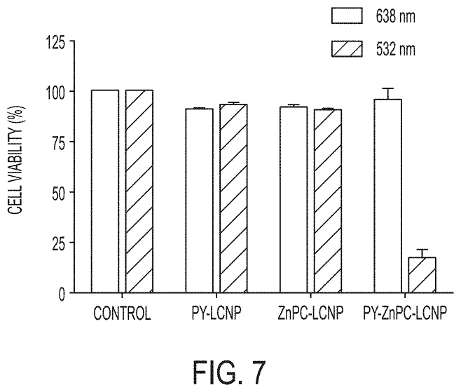

To further confirm these results, cells treated with PY-ZnPC-LCNPs (excited at 532 nm) were stained with trypan blue (a dye that is excluded by viable cells but taken up readily by non-viable cells). Compared to control, the cells treated with PY-ZnPC-LCNPs (excited at 532 nm) showed robust (nearly 100%) trypan blue staining, indicative of efficient cell killing by the LCNPs (FIG. 6C). As shown in FIG. 7, cell viability of Hela cell 72 h post-treatment using PY-ZnPC-LCNPs excited at 532 nm is only ˜20% which is significantly lower than that of ˜90% for PY-ZnPC-LCNPs (638 nm excitation) or ZnPC-LCNPs (638 nm or 532 nm excitation). Collectively, these results suggest that the efficient ROS generation via FRET excitation of ZnPC induces higher phototoxicity to cells, most likely due to the lipid peroxidation in the plasma membrane during the treatment which is directly facilitated by the close tethering of the LCNP by the PEG-Chol moiety.

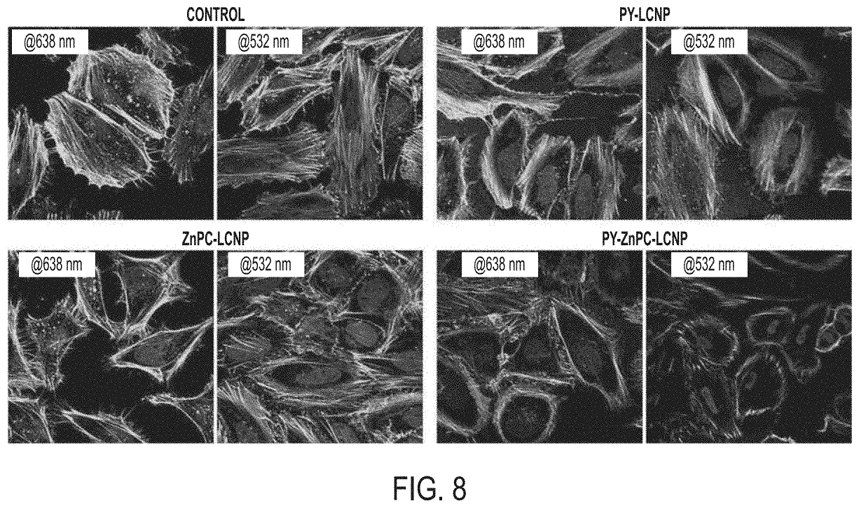

Finally, to understand the mechanism of the phototoxicity responses, the morphological change of the cell in early stages (2 h window) after the PDT treatment was studied by imaging cytosolic actin microfilaments and the nucleus. As shown in FIG. 8, control samples treated with only lasers 638 nm, 532 nm or PY-LCNPs with excitation at 638 nm or 532 nm maintained perfectly organized actin microfilaments and robust nuclear morphology. Conversely, cells treated with ZnPC-LCNPs (638 nm excitation) and PY-ZnPC-LCNPs (excitation at 638 nm or 532 nm excitation) showed a clear retraction of actin microfilaments to the cell periphery. Particularly, a significant change in actin microfilaments organization and nuclear morphology for the cells treated with PY-ZnPC-LCNPs (532 nm excitation) was observed. This is consistent with our observations from the proliferation studies described above.

### Further Embodiments

Potential uses of the invention include the use and sale of the material for cancer treatment without the need for invasive surgery for removing tumor. Other applications include dermatology (acne), ophthalmology (age-related macular degeneration), urology (bladder cancer), gastroenterology (stomach and esophageal cancer), and respiratory medicine (lung cancer). These materials also could find use in fluorescence image-based diagnosis of the tumor status/progression after treatment.

The LCNP could easily serve as host to other dye donor-acceptor pairs; conceivably increasing the water solubility of the dyes in the context of the LCNP carrier.

The surface of the LCNP can be decorated or conjugated with other biologicals (antibodies, proteins, peptides, small molecules, drugs) to facilitate targeting to specific cell types or subcellular structures.

Advantages

Use of the LCNP-PEG-Chol carrier (hydrophilic surface/hydrophobic core) as a host for the water-insoluble ZnPC provides water solubility and targeted (cell membrane) delivery of the ZnPC. This significantly increases the efficacy of the ZnPC PS moiety.

The ordered, crosslinked hydrophobic LCNP core and co-encapsulation of PY/ZnPC reduce the self-aggregation of the ZnPC, which minimizes its unfavorable optical quenching for PDT application.

The excitation of the ZnPC in a FRET configuration using the PY dye as the FRET energy donor facilitates significantly higher emission efficiency and ROS generation, a non-obvious outcome.

The bright emission profile of PY in LCNP facilitates the optical tracking of PY-ZnPC-LCNP during and after the PDT. Therefore, this preparation enables its eventual use in theranostic (combined diagnostic and therapeutic) applications.

The large two photon absorption (TPA) of the PY moiety (coupled with its ability to serve as a highly efficient FRET donor to the ZnPC acceptor) allows efficient excitation of the ZnPC using longer wavelength light that has higher tissue penetration. The ZnPC alone has minimal TPA, so the PY facilitates use of ZnPC in a two photon mode.

Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### References

Synthesis of LCNP and PEG-Chol Conjugation onto the Surface

Nag, O. K., et al., Lipid Raft-Mediated Membrane Tethering and Delivery of Hydrophobic Cargos from Liquid Crystal-Based Nanocarriers. Bioconjugate Chem. 2016. 27(4): 982-993.

Nag, O. K., et al., Targeted Plasma Membrane Delivery of a Hydrophobic Cargo Encapsulated in a Liquid Crystal Nanoparticle Carrier. J. Vis Exp. 2017. (120).

Spillmann, C. M., et al., Multifunctional Liquid Crystal Nanoparticles for Intracellular Fluorescent Imaging and Drug Delivery. ACS Nano, 2014. 8(7): 6986-6997.

Zhou, J. C., et al., Tuning mechanical properties of liquid crystalline nanoparticles. J. Colloid Interface Sci. 2012. 368(1): 152-157.

Spillmann, C. M., et al., Role of Surfactant in the Stability of Liquid Crystal-Based Nanocolloids. Langmuir, 2009. 25(4): 2419-2426.

ZnPC and ZnPC Loaded NPs for PDT

Kim, J., et al., Selective photosensitizer delivery into plasma membrane for effective photodynamic therapy. J. Control Release, 2014. 191: 98-104.

Ping, J.-t., et al., Synthesis and optimization of ZnPc-loaded biocompatible nanoparticles for efficient photodynamic therapy. J. Mater. Chem. B. 2016. 4(25): 4482-4489.

Makhseed, S., et al., Water-soluble non-aggregating zinc phthalocyanine and in vitro studies for photodynamic therapy. Chem. Commun. 2013. 49(95): 11149-11151.

Lucky, S. S., et al., Nanoparticles in photodynamic therapy. Chem. Rev. 2015. 115(4): 1990-2042.

Feuser, P. E., et al., Synthesis of ZnPc loaded poly(methyl methacrylate) nanoparticles via miniemulsion polymerization for photodynamic therapy in leukemic cells. Mater. Sci. Eng. C. 2016. 60: 458-466.

Kiew, L. V., et al., Near-infrared activatable phthalocyanine-poly-L-glutamic acid conjugate: increased cellular uptake and light-dark toxicity ratio toward an effective photodynamic cancer therapy. Nanomedicine, 2017. 13(4): 1447-1458.

Jin, Y, et al., Nanostructures of an amphiphilic zinc phthalocyanine polymer conjugate for photodynamic therapy of psoriasis. Colloids Surf. B. Biointerfaces, 2015. 128: 405-409.

Li, L., et al., Enhanced photodynamic efficacy of zinc phthalocyanine by conjugating to heptalysine. Bioconjugate Chem. 2012. 23(11): 2168-2172.

Drozdek, S., et al., Two-Photon Induced Fluorescence Energy Transfer in Polymeric Nanocapsules Containing CdSexS1-x/ZnS Core/Shell Quantum Dots and Zinc(II) Phthalocyanine. J. Phys. Chem. C. 2016. 120(28): 15460-15470.

What is claimed is:

1. A nanoparticle comprising:
a liquid crystal nanoparticle (LCNP) encapsulating perylene and Zinc (II) phthalocyanine.

2. The nanoparticle of claim 1, wherein the nanoparticle is in a state of having been made by reacting an acrylate liquid crystal cross-linking agent and a carboxyl-terminated polymerizable surfactant.

3. The nanoparticle of claim 1, wherein the LCNP is functionalized for targeting of cellular membranes.

4. The nanoparticle of claim 3, wherein the LCNP is conjugated to a poly(ethylene glycol)-cholesterol compound.

5. A nanoparticle comprising:
a liquid crystal nanoparticle (LCNP) encapsulating perylene and Zinc (II) phthalocyanine;
wherein the nanoparticle is in a state of having been made by reacting an acrylate liquid crystal cross-linking agent and a carboxyl-terminated polymerizable surfactant; and
wherein the LCNP is conjugated to a poly(ethylene glycol)-cholesterol compound.

6. A method of delivery comprising:
providing a liquid crystal nanoparticle (LCNP) encapsulating perylene and Zinc (II) phthalocyanine;
delivering the LCNP to mamallian cells; and
optically exciting the ZnPC via fluorescence resonance energy transfer (FRET) by using the photoexcited PY as the energy donor, thereby generating reactive oxygen species (ROS).

7. The method of claim 6, wherein the nanoparticle is in a state of having been made by reacting an acrylate liquid crystal cross-linking agent and a carboxyl-terminated polymerizable surfactant.

8. The method of claim 6, wherein the LCNP is functionalized for targeting of cellular membranes.

9. The method of claim 8, wherein the LCNP is conjugated to a poly(ethylene glycol)-cholesterol compound.
